(19) United States
(12) Patent Application Publication
(10) Pub. No.: US 2018/0214065 A1
(43) Pub. Date: Aug. 2, 2018

(54) Allergy Skin Testing Devices with Compressible Annular Pain-Reduction Structures

(71) Applicants: Richard Caizza, Union City, NJ (US); Ziv Harish, Tenafly, NJ (US); Russ Weinzimmer, Milford, NH (US)

(72) Inventors: Richard Caizza, Union City, NJ (US); Ziv Harish, Tenafly, NJ (US); Russ Weinzimmer, Milford, NH (US)

(21) Appl. No.: 15/419,738

(22) Filed: Jan. 30, 2017

(51) Int. Cl. A61B 5/00

(52) U.S. Cl. CPC A61B 5/411

(57) ABSTRACT

An allergy skin testing device includes a central post having a sharp portion, a longitudinal portion, and a guide portion. The sharp portion has one or more sharp probes extending longitudinally. The guide portion facilitates guiding the sharp portion towards the skin. The device also includes a compressible annular pain-reduction structure having an annular dull probe portion, a resilient compressible portion, and an optional base portion. The annular dull probe portion initially extends beyond the sharp portion, and has a passageway allowing passage of the sharp portion therethrough, the annular dull probe portion being configured to activate a surrounding pain gate effect (a “pain gate fence”) when pressed against the skin. The resilient compressible portion is configured to compress along the longitudinal portion of the central post. The base portion is configured to be pushed by the guide portion when the annular dull probe portion is pressed against the skin.

### FIELD OF THE INVENTION

This invention relates generally to allergy skin testing devices, and particularly to allergy skin testing devices with structures to reduce pain.

### BACKGROUND OF THE INVENTION

Traditional allergy skin testing devices inflict pain while introducing allergens to the skin using sharp probes. According to pain-gate theory, applying dull pressure shortly before the sharp probes penetrate the skin to introduce the allergens reduces perception of the pain caused by the sharp probes. Reduced-pain allergy skin testing devices employing pain-gate theory to allergy skin testing were first taught by Harish et al. For example, Harish et al first taught using a single dull probe for each plurality of sharp probes. Heine et al then taught a version of this approach using a single dull probe for each plurality of surrounding sharp probes. However, each single dull probe of Heine et al does not provide an acceptable level of pain reduction for the corresponding plurality of surrounding sharp probes.

### SUMMARY OF THE INVENTION

According to the invention, an annular surface of a Compressible Annular Pain-Reduction Structure is pressed against the skin before one or more sharp probes is pressed into the skin surrounded by the annular surface so as to deliver an allergen to the skin for testing purposes, the pain typically caused by the one or more sharp probes being blocked by the pain gating effects of the Compressible Annular Pain-Reduction Structure. The Compressible Annular Pain-Reduction Structure provides a surround-effect that creates a plurality pain gates substantially surrounding an area of sharp probe penetration that would be painful if not blocked by the surrounding pain gates. The plurality of surrounding pain gates is referred to as PAIN FENCE™ pain reduction technology. Thus, a PAIN FENCE™ is any arrangement of pain-gates that substantially block or inhibit pain signals that tend to not be blocked by a single dull probe.

The Compressible Annular Pain-Reduction Structure also tends to visually hide the one or more sharp probes surrounded thereby, as like being hidden behind a fence, which makes the skin testing device of the invention less intimidating, and consequently less upsetting, to children.

Further, by avoiding upsetting a child to be tested by the device, pain perception is further reduced beyond the physiological “pain gate” effect caused by the surrounding Compressible Annular Pain-Reduction Structure.

In addition, the physician or nurse administering the test can suggest to the patient to expect to feel a dull pressure, such as by saying “you will feel a dull pressure”. Since the patient only sees the Compressible Annular Pain-Reduction Structure (which effectively hides the sharp probes inside), and has been told to expect to “feel a dull pressure”, he/she fully expects to feel dull pressure, and so is even less likely to experience any discomfort.

A general aspect of the invention is an allergy skin testing device which is to be used by pressing the device against skin. The device includes: a central post having a sharp portion, a longitudinal portion, and a guide portion, the sharp portion having one or more sharp probes extending longitudinally and being configured to administer an allergen to the skin, the guide portion being configured to facilitate guiding the sharp portion towards the skin, the longitudinal portion extending between the sharp portion and the guide portion; and a compressible annular pain-reduction structure having an annular dull probe portion, a resilient compressible portion, and a base portion, the annular dull probe portion initially extending beyond the sharp portion of the central post, and having a passageway allowing passage of the sharp portion therethrough, the annular dull probe portion being configured to activate a surrounding pain gate effect when pressed against the skin, the resilient compressible portion being configured to compress along the longitudinal portion of the central post while the annular dull probe portion is pressed against the skin, the base portion being configured to be pushed by the guide portion of the central post when the annular dull probe portion is pressed against the skin, thereby compressing the resilient compressible portion until the one or more sharp probes are pressed into the skin so as to administer the allergen to the skin.

In some embodiments, the resilient compressible portion of the compressible annular pain-reduction structure includes: a plurality of bendable elements configured to resiliently bend when compressed.

In some embodiments, the resilient compressible portion of the compressible annular pain-reduction structure includes: a single bendable element configured to resiliently bend when compressed.

In some embodiments, the resilient compressible portion of the compressible annular pain-reduction structure includes: a bellows configured to resiliently shorten when compressed.

In some embodiments, the resilient compressible portion of the compressible annular pain-reduction structure includes: a spring configured to resiliently shorten when compressed.

In some embodiments, the annular dull probe portion has a C-shaped cross-section.

In some embodiments, the annular dull probe portion includes one or more bumps on an annular surface of the annular dull probe portion that contacts the skin.

In some embodiments, the device further includes: a sealing ring seated at a point along the sharp portion of the central post, the sealing ring being in slidable contact with an inner surface of the passageway of the annular dull probe portion so as to ensure that the sharp probes of the sharp portion contact the skin in substantial concentric relationship with the annular contact surface of the annular dull probe portion that contacts the skin.

In some embodiments, the device further includes: a friction-free coating applied to the inner surface of the passageway so as to reduce friction between the sealing ring and the inner surface of the passageway.

In some embodiments, the guide portion of the central post includes an attachment feature for attaching the guide portion to a handle of a multiple test allergy skin testing device. In further embodiments, the device also includes a handle of a multiple test allergy skin testing device.

Another general aspect of the invention is a multiple test allergy skin testing device, that is used by pressing the device against skin. This device includes: a plurality of central posts, each central post having a sharp portion, a longitudinal portion, and a guide portion, the sharp portion having one or more sharp probes extending longitudinally and being configured to administer an allergen to the skin, the guide portion being configured to facilitate guiding the sharp portion towards the skin, the longitudinal portion extending between the sharp portion and the guide portion; a plurality of compressible annular pain-reduction structures, each compressible annular pain-reduction structure having an annular dull probe portion, a resilient compressible portion, and a base portion, the annular dull probe portion initially extending beyond the sharp portion of the central post, and having a passageway allowing passage of the sharp portion therethrough, the annular dull probe portion being configured to activate a surrounding pain gate effect when pressed against the skin, the resilient compressible portion being configured to compress along the longitudinal portion of the central post while the annular dull probe portion is pressed against the skin, the base portion being configured to be pushed by the guide portion of the central post when the annular dull probe portion is pressed against the skin, thereby compressing the resilient compressible portion until the one or more sharp probes are pressed into the skin so as to administer the allergen to the skin; and a gripping element configured to receive the plurality of central posts, the gripping element being for securely controlling the plurality of central posts and the respective plurality of compressible annular pain-reduction structures, so as to facilitate pressing the annular dull probe portion of each compressible annular pain-reduction structure, and then the sharp portion of each central post, into the skin using one press of the gripping element towards the skin.

In some embodiments, the resilient compressible portion of each compressible annular pain-reduction structure includes: a pair of bendable elements configured to resiliently bend when compressed.

In some embodiments, the resilient compressible portion of each compressible annular pain-reduction structure includes: a bellows configured to resiliently shorten when compressed.

In some embodiments, the resilient compressible portion of each compressible annular pain-reduction structure includes: a spring configured to resiliently shorten when compressed.

In some embodiments, each annular dull probe portion has a C-shaped cross-section.

In some embodiments, each annular dull probe portion includes one or more bumps on an annular surface of the annular dull probe portion that contacts the skin.

In some embodiments, the device further includes: a sealing ring seated at a point along the sharp portion of the central post, the sealing ring being in slidable contact with an inner surface of the passageway of the annular dull probe portion so as to ensure that the sharp probes of the sharp portion contact the skin in substantial concentric relationship with the annular contact surface of the annular dull probe portion that contacts the skin. In further embodiments, the device also includes: a friction-free coating applied to the inner surface of the passageway so as to reduce friction between the sealing ring and the inner surface of the passageway.

In some embodiments, the guide portion of each central post includes an attachment feature for attaching the guide portion to the gripping element.

Another general aspect of the invention is an allergy skin testing device that is used by pressing the device against skin, the device including: a central post having a sharp portion, a longitudinal portion, and a guide portion, the sharp portion having one or more sharp probes extending longitudinally and being configured to administer an allergen to the skin, the guide portion being configured to facilitate guiding the sharp portion towards the skin, the longitudinal portion extending between the sharp portion and the guide portion; and a compressible annular pain-reduction structure having an annular dull probe portion, and a resilient compressible portion, the annular dull probe portion initially extending beyond the sharp portion of the central post, and having a passageway allowing passage of the sharp portion therethrough, the annular dull probe portion being configured to activate a surrounding pain gate effect when pressed against the skin, the resilient compressible portion being configured to compress along the longitudinal portion of the central post while the annular dull probe portion is pressed against the skin.

In some embodiments, the compressible annular pain-reduction structure further includes: a cover portion that covers the resilient compressible portion, the cover portion being attached to the annular dull probe portion of the compressible annular pain-reduction structure.

In some embodiments, the resilient compressible portion of the compressible annular pain-reduction structure includes: a plurality of bendable elements configured to resiliently bend when compressed.

In some embodiments, the resilient compressible portion of the compressible annular pain-reduction structure includes: a single bendable element configured to resiliently bend when compressed.

In some embodiments, the resilient compressible portion of the compressible annular pain-reduction structure includes: a bellows configured to resiliently shorten when compressed.

In some embodiments, the resilient compressible portion of the compressible annular pain-reduction structure includes: a spring configured to resiliently shorten when compressed.

In some embodiments, the annular dull probe portion includes one or more bumps on an annular surface of the annular dull probe portion that contacts the skin.

In some embodiments, the device further includes: a friction-free coating applied to the inner surface of the passageway so as to reduce friction between the sharp portion and an inner surface of the passageway.

In some embodiments, the guide portion of the central post includes an attachment feature for attaching the guide portion to a handle of a multiple test allergy skin testing device.

In some embodiments, the device further includes a handle of a multiple test allergy skin testing device.

### BRIEF DESCRIPTION OF THE DRAWINGS

The invention will be more fully understood from the following detailed description, in conjunction with the following figures, wherein:

FIG. 1 is a isometric view of an embodiment of the allergy skin testing device.

FIG. 2 is a side view of the allergy skin testing device of FIG. 1.

FIG. 3 is a cross-sectional side view of the allergy skin testing device of FIGS. 1 and 2.

FIG. 4 is an isometric view of a plurality of the allergy skin testing device of FIGS. 1, 2, and 3 joined together by a handle so as to form a multiple test allergy skin testing device.

FIG. 5 is a bottom view of the multiple test allergy skin testing device of FIG. 4.

FIG. 6 is an isometric view of the multiple test allergy skin testing device pressed against a skin surface and responding to downward pressure on the handle by entering a compressed state.

FIG. 7 is an isometric view of the multiple test skin testing device of FIG. 6 in a compressed state, showing the sharps probes extending through and surrounded by the annular dull probe.

FIG. 8 is an isometric view of the allergy skin testing device of FIG. 1 in a compressed state.

FIG. 9 is an exploded view of the allergy skin testing device of FIG. 1.

FIG. 10 is an isometric view of an alternate embodiment of the skin testing device having a resilient compressible portion with only a single bendable element.

FIG. 11 is an isometric view of the alternate embodiment of the skin testing device of FIG. 10 in a compressed state, showing the sharps probes extending through and surrounded by an annular surface of the annular dull probe.

FIG. 12 is an isometric view of yet another alternate embodiment of the skin testing device having a slotted annular dull probe, the slotted annular dull probe having one or more contact bumps.

FIG. 13 is an isometric view of still another alternate embodiment of the skin testing device having a bellows as the compression member.

FIG. 14 is a translucent isometric view of the embodiment of FIG. 13.

FIG. 15 is an translucent isometric view of still another alternate embodiment of the skin testing device having a sprint as the compression member.

FIG. 16 is a side view of the embodiment of the allergy skin testing device of FIG. 2, further including a sealing ring.

FIG. 17 is a translucent isometric view of the embodiment of the skin testing device of FIG. 10 having a resilient compressible portion with only a single bendable element, also including a pair of stabilizing sealing rings.

FIG. 18 is a cross-sectional side view of yet another alternate embodiment of the skin testing device having a spring and a cover that extends over the spring.

FIG. 19 is a side view of the embodiment of FIG. 18, showing the annular dull probe contacting the skin before downward force is applied to the guide portion.

### DETAILED DESCRIPTION

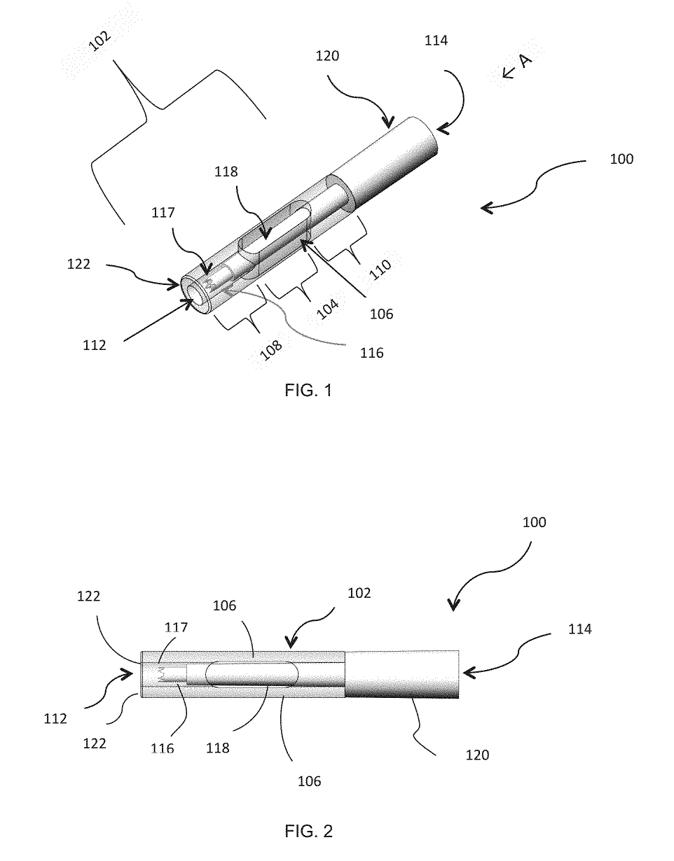

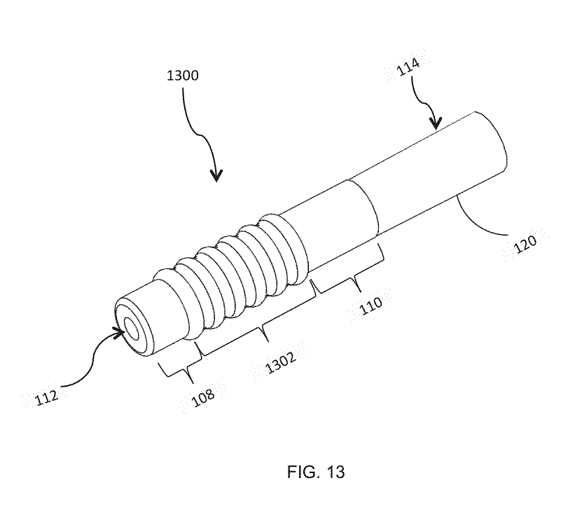

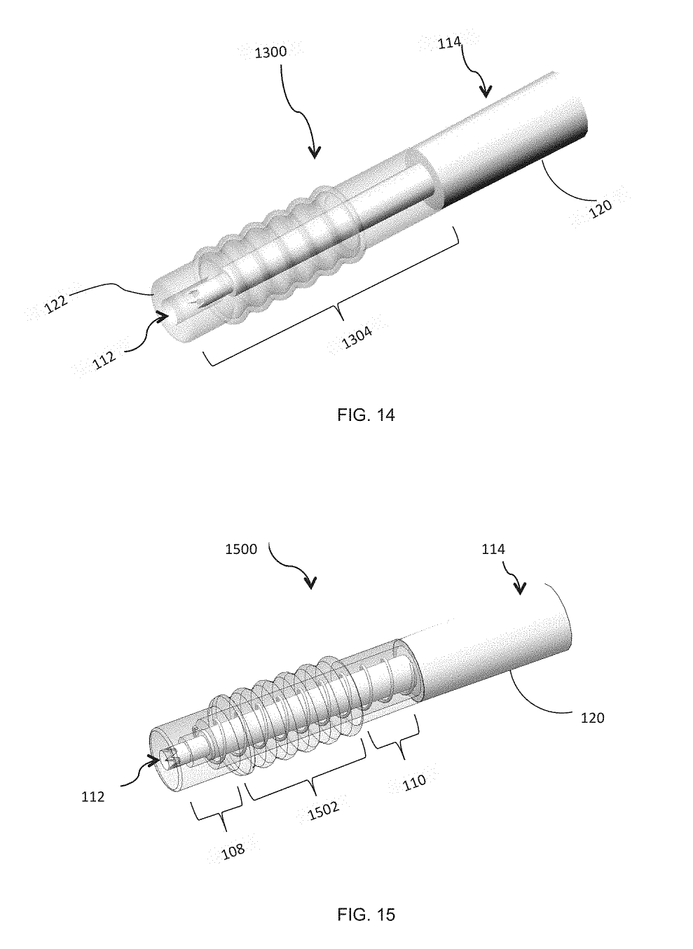

With reference to FIGS. 1 and 2, an embodiment of an allergy skin testing device 100 is shown having a compressible annular pain-reduction structure 102. In this embodiment 100, the resilient compressible portion 104 of the compressible annular pain-reduction structure 102 includes a pair of bendable elements 106 configured to resiliently bend when compressed. Note that the pair of bendable elements 106 could also be three, four, or five bendable elements, for example. The compressible annular pain-reduction structure 102 also includes an annular dull probe portion 108, and a base portion 110. The compressible annular pain-reduction structure 102 can be made from a simple extruded tube-like structure, such as shown in FIGS. 1-12, or can be a bellows-like structure, such as shown in FIGS. 13-14. The compressible annular pain-reduction structure 102 can be made of a thermoplastic, natural or synthetic rubber, or metal, for example.

Extending through a passageway 112 of the compressible annular pain-reduction structure 102 is a central post 114 having a sharp portion 116, a longitudinal portion 118, and a guide portion 120. The sharp portion 116 has one or more sharp probes 117 extending longitudinally and being configured to administer an allergen to the skin. The guide portion 120 is configured to facilitate guiding the sharp portion 116 towards the skin. The longitudinal portion 118 extends between the sharp portion 116 and the guide portion 120. The central post can be made from plastic, composite, or other light-weight material.

The annular dull probe portion 108 initially extends beyond the sharp portion 116 of the central post 114, and has a passageway 112 that allows passage of the sharp portion 116 therethrough when the guide portion 120 is pressed towards the skin. The annular dull probe portion 108 is configured to activate a surrounding pain gate effect (also called a Pain Fence™) when the annular surface 122 of the annular dull probe portion 108 is pressed against the skin.

The resilient compressible portion 104 is configured to compress along the longitudinal portion of the central post 114 while the annular surface 122 of the annular dull probe portion 108 is pressed against the skin. Thus, the resilient compressible portion 104 provides a pushing force to the annular dull probe portion 108 while the sharp probes 117 of the sharp portion 116 penetrate the skin enough to introduce the allergen that adheres to the sharp probes 117.

The base portion 110 of the compressible annular pain-reduction structure 102 is configured to be pushed by the guide portion 120 of the central post 114 when the annular dull probe portion 108 is pressed against the skin, thereby compressing the resilient compressible portion 104 until the one or more sharp probes 117 are pressed into the skin so as to administer the allergen to the skin. As the base portion 110 is pulled away from the skin by the guide portion 120 of the central post 114, the resilient compressible portion 104 expands, the sharp probes 117 move away from the skin, and then the annular dull probe portion 108 moves away from the skin.

In a preferred embodiment, reduction of pain was demonstrated when annular dull probe portion 108 extended beyond the sharp probes 117 by 1/16 inch, and the pair of bendable elements 106 resisted buckling until a force of 1-2 pounds was applied. The annular surface 122 of the annular dull probe portion 108 measured ¼ inch in diameter, with a wall thickness of 1/16 inch. The wall thickness is uniform all along the compressible annular pain-reduction structure 102, from the annular surface 122 of the annular dull probe portion 108, all the way to the base portion 110, including the bendable elements 106.

In this preferred embodiment, the bendable elements 106 are ½ inch long by 1/16 inch wide. As the bendable elements 106 buckle, the sharp probes 117 move through the passageway 112 to make contact with the skin, delivering the allergen to be tested thereto.

In this preferred embodiment, the material used for the bendable elements 106 is silicone with a hardness between 30-40 Shore A. The material tested was between 30-40 Shore A. “Shore A” is a hardness scale used to quantify hardness of rubber and plastics. Other materials can also be used, such as thermoplastic elastomer (TPE), polyurethane (PU), polyethylene (PE), natural rubber, or synthetic rubber, for example.

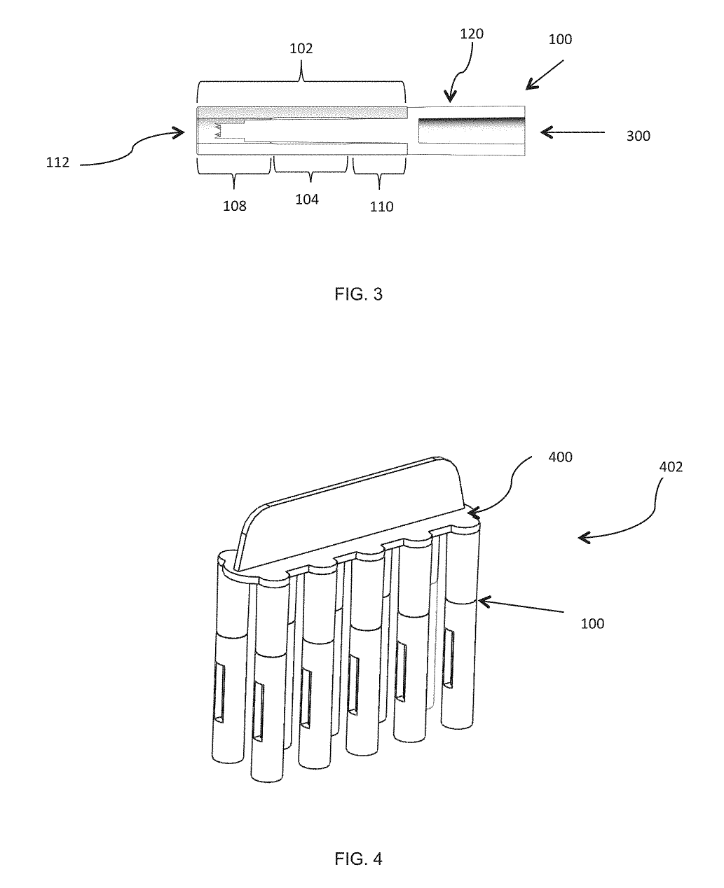

FIG. 3 shows a cross-sectional side view of the allergy skin testing device of FIGS. 1 and 2, further showing the guide portion 120 having a slot 300 which can serve as an attachment feature for attaching the guide portion 120 to a handle of a single allergy testing device, or to a handle 400 of multiple allergy testing device 402 having ten allergy testing devices 100, for example as shown in FIG. 4.

FIG. 4 shows a plurality of the allergy skin testing devices 100 of FIG. 1, joined together by a handle 400 so as to form a multiple allergy skin testing device 402.

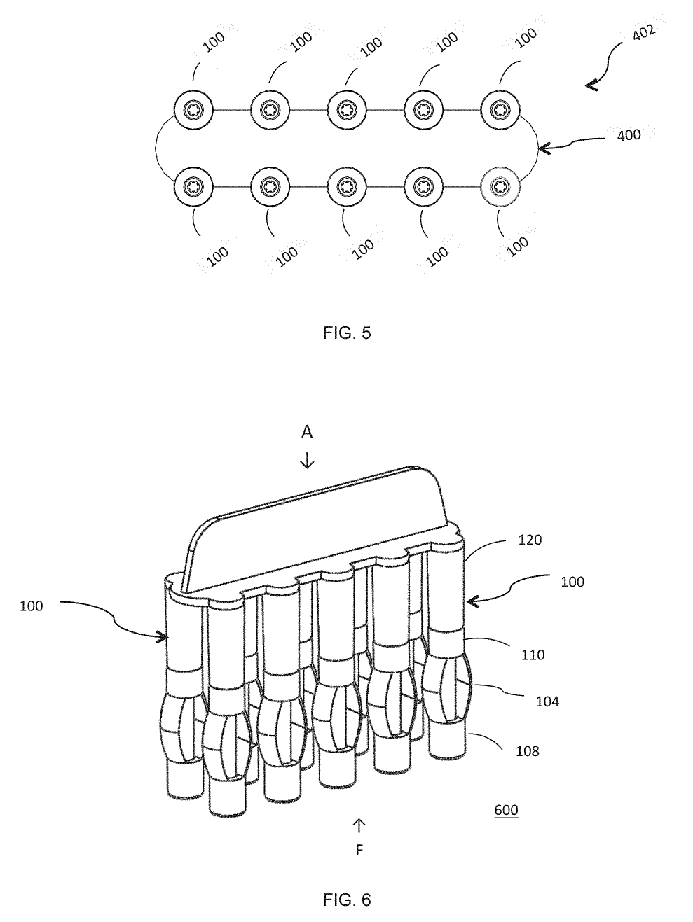

FIG. 5 shows a view from below of the plurality of the allergy skin testing devices 100 of FIG. 1, joined together by a handle 400 so as to form a multiple allergy skin testing device 402.

FIG. 6 shows the multiple allergy skin testing device 402 being pressed downward against skin 600 with force A applied to the handle 400, causing a reacting force F upward from the skin 600 as the device 402 is pressed against the skin 600 of a person being tested for various allergies. The force A is applied until the sharp probes 117 press into the skin 600 enough to transfer some allergen to the skin 600. Note that the sharp probes 117 cannot be seen by the person being tested because the sharp probes 117 are blocked from view by the annular dull probe portion 108. Also note that the resilient compressible portion 104 bows outward for each device 100 when the force A is applied to the handle 400. The handle 400 applies the force A to both the guide portion 120, and to the base portion 110. The annular dull probe portion 108 presses against the skin 600 while the sharp probes 117 press into the skin 600, the pressure of the annular dull probe portion 108 causing a surrounding pain gate effect in the nerves surrounding the points within receiving the sharp probes 117, substantially reducing the perception of pain due to the sharp probes 117.

When force A is reversed, as when the device 402 is pulled away from the skin 600, the sharp probes 117 retreat back into the passageway 112 of the annular dull probe portion 108. The annular dull probe portion 108 shields the sharp probes 117 from sight, thereby reducing anxiety of the patients who may fear sharp probes.

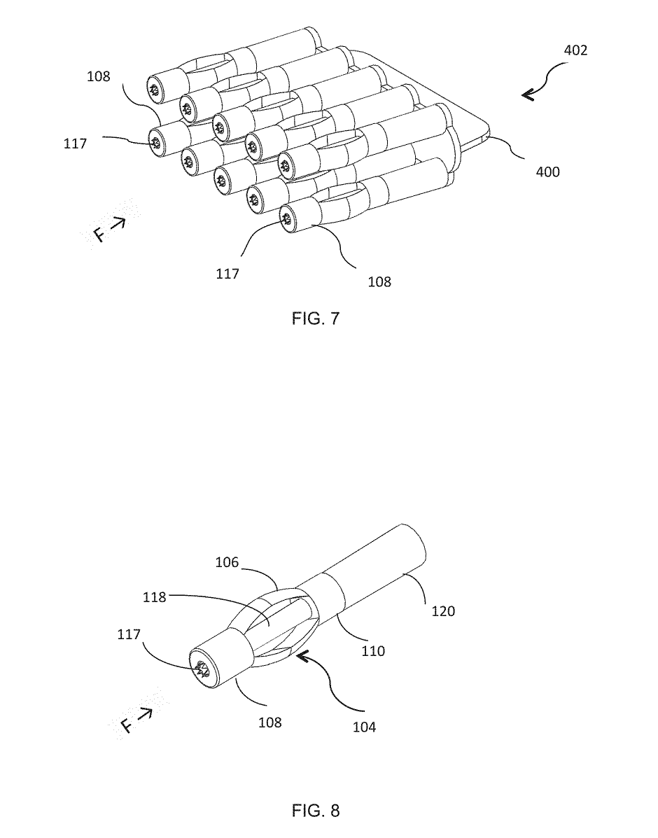

FIG. 7 shows a bottom and side view of the device 402 of FIG. 6, also in the compressed state, but now showing the sharp probes 117 emerging from the passageway 112 of each annular dull probe portion 108.

FIG. 8 shows a single tester device 100 of the multiple allergy skin testing device 402 of FIG. 6, also in a compressed state due to application of the force F. The sharp probes 117 can be seen extending beyond the annular dull probe portion 108 through the passageway 112. The bendable elements 106 of the resilient compressible portion 104 are also seen flanking the longitudinal portion 118 that extends between the sharp portion 116 supporting the sharp probes 117 and the guide portion 120.

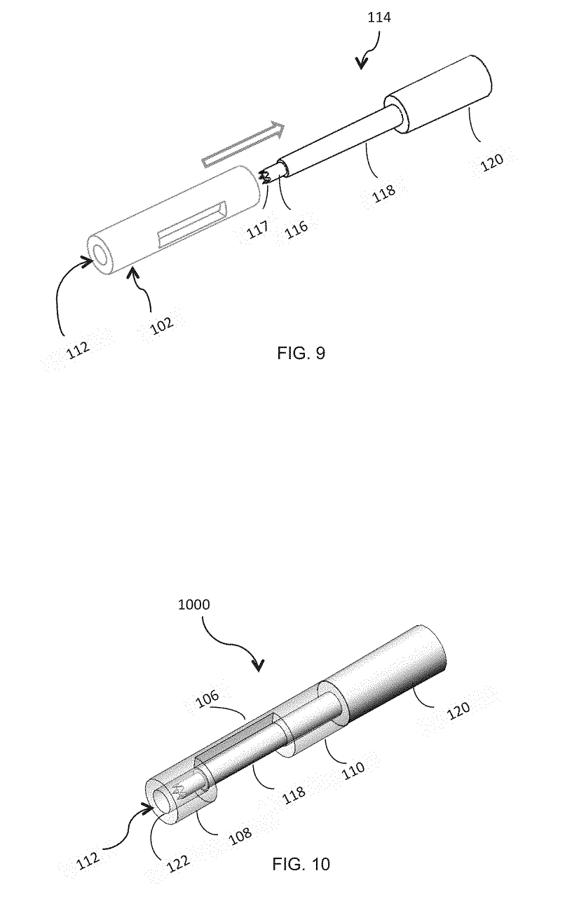

FIG. 9 is an exploded view of the allergy skin testing device of FIG. 1, showing the central post 114 having a sharp portion 116, a longitudinal portion 118, and a guide portion 120. The sharp portion 116 has one or more sharp probes 117 extending longitudinally and is configured to administer an allergen to the skin. The guide portion 120 is configured to facilitate guiding the sharp portion 116 towards the skin. The longitudinal portion 118 extends between the sharp portion 116 and the guide portion 120. The sharp portion 116 and the longitudinal portion 118 extend through most of the compressible annular pain-reduction structure 102 while the compressible annular pain-reduction structure 102 is not compressed, as shown in FIG. 2.

FIG. 10 is an isometric view of an alternate embodiment 1000 of the skin testing device having an asymmetric resilient compressible portion 104. In this embodiment 1000, the resilient compressible portion 104 has only a single bendable element 106 configured to resiliently bend when compressed.

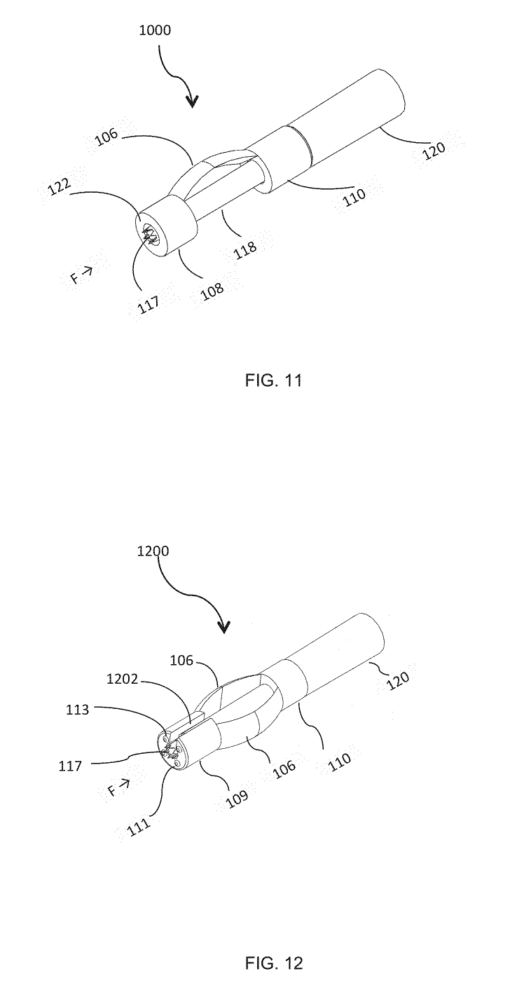

FIG. 11 shows the alternate embodiment 1000 of FIG. 10 in a compressed state, showing the sharps probes 117 extending through and surrounded by the annular surface 122 of the annular dull probe portion 108.

FIG. 12 is an isometric view of yet another alternate embodiment 1200 of the skin testing device having a slotted annular dull probe portion 109. A slot 1202 runs longitudinally along the slotted annular dull probe portion 109. The annular surface 111 of the slotted annular dull probe portion 109 has one or more bumps 113. Thus, the annular dull probe portion 109 need not fully surround the sharp probes to provide a surface for providing a surrounding pain gate effect. Further, the annular surface 111 of the annular dull probe portion 109 that contacts a patient's skin may include surface features, such as bumps 113 or ridges, to distract the patient from any sensation of the sharp probes 117 penetrating the skin, and to enhance the surrounding pain gate effect.

FIG. 13 is an isometric view of still another alternate embodiment 1300 of the skin testing device having an accordion resilient compressible portion 1302 between the annular dull probe portion 108, and the base portion 110. In this view, the guide portion 120 of the central post 114 is visible. Note that the sharp portion 116 is not visible within the passageway 112.

FIG. 14 is a translucent isometric view of the embodiment of FIG. 13. In this view, the entire central post 114 is visible, including the sharp portion 116 and the longitudinal portion 118. The sharp portion 116 and the longitudinal portion 118 extend through the entire compressible annular pain-reduction structure 1304, and will extend beyond the annular surface 122 and out from the passage way 112 when the resilient compressible portion 1302 is compressed.

FIG. 15 is a translucent isometric view of still another alternate embodiment 1500 of the skin testing device having a spring resilient compressible portion 1502 between the annular dull probe portion 108, and the base portion 110. In this view, the guide portion 120 of the central post 114 is visible. Note that the sharp portion 116 is visible within the passageway 112, and will emerge from the passageway 112 when the spring resilient compressible portion 1502 is compressed. The spring resilient compressible portion 1502 can include helical metal wire, helical plastic, or other resilient material. The spring resilient compressible portion 1502 can extend through the base portion 110. The spring resilient compressible portion 1502 can include an optional bellows cover portion like the bellows 1302 in FIG. 13. The annular dull probe portion 108, and the base portion 110 can be made from a thermoplastic, medical-grade plastic, a natural or synthetic rubber, silicone, or metal, for example.

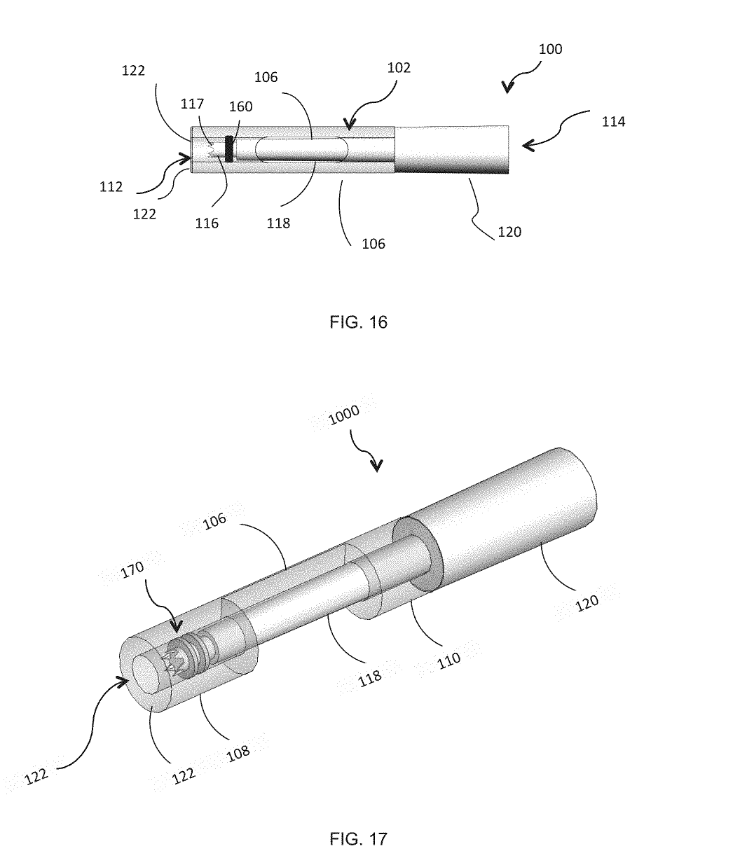

FIG. 16 is a side view of the allergy skin testing device 100 of FIG. 2, further including a sealing ring 160 seated in a circumferential notch in the sharp portion 116 for providing a sliding seal against the interior surface of the passageway 112 of the annular dull probe portion 108. The sealing ring 160 can be a substantially friction-free configuration of a sealing ring, such as an o-ring, or a piston seal with a z- or v-shaped cross-section. This type of sealing ring can be coated with a friction-free coating, such as Slik-Sil® from Surface Solutions Group, Chicago, Ill., or parylene types N, C, or D, internal lubricated silicone, or a dry lubricant, or similar coating to achieve substantially friction-free performance that facilitates easy and smooth longitudinal movement of the sharp portion 116 of the central post 114 within the passageway 112 of the annular dull probe portion 108. Alternatively, the sealing ring 160 can be used without a friction-free coating. The sealing ring 160 ensures that the sharp probes 117 will contact the skin within an area that is substantially radially equidistant from the annular surface 122 of the dull probe portion 108, resulting in a more consistent surrounding pain gate effect. Without a sealing ring 160, even slight lateral movements of the device 100 while pressing the device 100 against the skin can result in the sharp probes 117 contacting the skin off-center with respect to the annular surface 122 of the dull probe portion 108, possibly resulting in a diminished surrounding pain gate effect for at least some of the sharp probes 117.

FIG. 17 is a translucent isometric view of the alternate embodiment 1000 of the skin testing device having a resilient compressible portion 104 with only a single bendable element 106, also including a pair of sealing rings 170 each seated in a respective circumferential notch in the sharp portion 116. The pair of sealing rings 170 provide a stabilizing sliding seal against the interior surface of the passageway 112 of the annular dull probe portion 108. The pair of sealing rings 117 provide added stability against bending forces due to the asymmetric resilient compressible portion 104 having only a single bendable element 106 configured to resiliently bend when compressed as the device is pressed against the skin. By absorbing at least some of the bending forces due to compression, the sharp probes 117 are more likely to contact the skin within an area that is radially equidistant from the annular surface 122 of the dull probe portion 108, resulting in a more consistent surrounding pain gate effect. As in the embodiment of FIG. 16, the pair of sealing rings 170 can be coated with a friction-free coating.

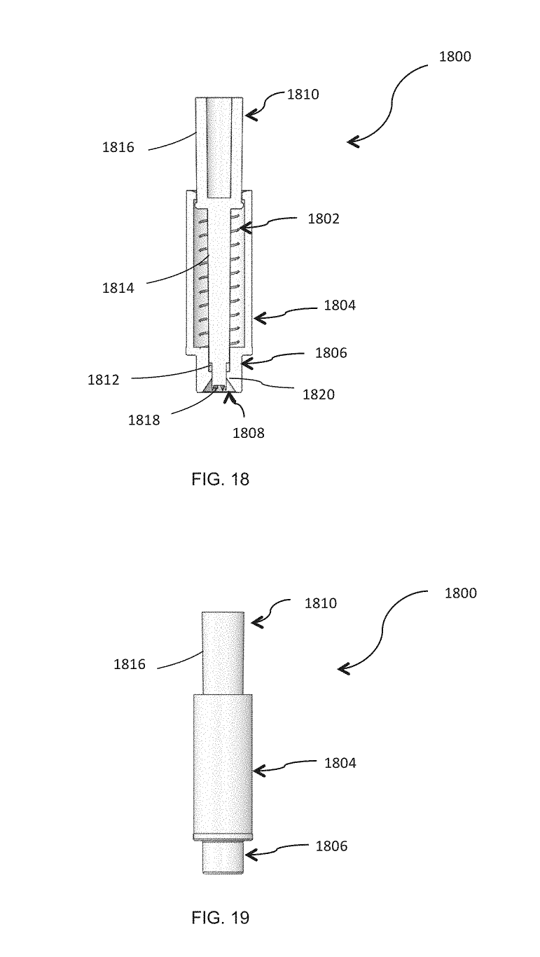

FIG. 18 is a cross-sectional side view of yet another alternate embodiment 1800 of the skin testing device having a spring 1802 serving as the resilient compressible portion, and a cover portion 1804 that extends over the spring 1802. The cover portion 1804 extends from the annular dull probe portion 1806 over the spring 1802.

Extending through a passageway 1808 is a central post 1810 having a sharp portion 1812, a longitudinal portion 1814, and a guide portion 1816. The sharp portion 1812 has one or more sharp probes 1818 extending longitudinally and being configured to administer an allergen to the skin.

Note that in this embodiment 1800, the sharp portion 1812 is maintained in concentric alignment with the annular dull probe portion 1806 by the narrowing 1820 having a slightly larger diameter than the diameter of the sharp portion 1812, such that they are in concentric sliding contact with each other. A friction-free coating, such as Slik-Sil® from Surface Solutions Group, Chicago, Ill., or parylene types N, C, or D, internal lubricated silicone, or a dry lubricant, or similar coating, can be used to achieve substantially friction-free relative movement.

FIG. 19 is a side view of the embodiment of FIG. 18, showing the annular dull probe portion 1806 contacting the skin before downward force is applied to the guide portion 1816. Downward force applied to the guide portion 1816 causes the entire central post 1810 to move downward and into the cover portion 1804, thereby compressing the spring 1802 inside, which in turn urges the annular dull probe portion 1806 to press into the skin surrounding the skin that is impinged upon by the sharps 1818, thereby activating a surrounding pain gate effect while the sharps introduce allergen to the skin.

Other modifications and implementations will occur to those skilled in the art without departing from the spirit and the scope of the invention as claimed. Accordingly, the above description is not intended to limit the invention, except as indicated in the following claims.

What is claimed is:

1. An allergy skin testing device, the device to be used by pressing the device against skin, the device comprising:
a central post having a sharp portion, a longitudinal portion, and a guide portion,
the sharp portion having one or more sharp probes extending longitudinally and being configured to administer an allergen to the skin,
the guide portion being configured to facilitate guiding the sharp portion towards the skin,
the longitudinal portion extending between the sharp portion and the guide portion; and
a compressible annular pain-reduction structure having an annular dull probe portion, a resilient compressible portion, and a base portion,
the annular dull probe portion initially extending beyond the sharp portion of the central post, and having a passageway allowing passage of the sharp portion therethrough, the annular dull probe portion being configured to activate a surrounding pain gate effect when pressed against the skin,
the resilient compressible portion being configured to compress along the longitudinal portion of the central post while the annular dull probe portion is pressed against the skin,
the base portion being configured to be pushed by the guide portion of the central post when the annular dull probe portion is pressed against the skin, thereby compressing the resilient compressible portion until the one or more sharp probes are pressed into the skin so as to administer the allergen to the skin.

2. The device of claim 1, wherein the resilient compressible portion of the compressible annular pain-reduction structure includes:
a plurality of bendable elements configured to resiliently bend when compressed.

3. The device of claim 1, wherein the resilient compressible portion of the compressible annular pain-reduction structure includes:
a single bendable element configured to resiliently bend when compressed.

4. The device of claim 1, wherein the resilient compressible portion of the compressible annular pain-reduction structure includes:
a bellows configured to resiliently shorten when compressed.

5. The device of claim 1, wherein the resilient compressible portion of the compressible annular pain-reduction structure includes:
a spring configured to resiliently shorten when compressed.

6. The device of claim 1, wherein the annular dull probe portion has a C-shaped cross-section.

7. The device of claim 1, wherein the annular dull probe portion includes one or more bumps on an annular surface of the annular dull probe portion that contacts the skin.

8. The device of claim 1, further comprising:
a sealing ring seated at a point along the sharp portion of the central post, the sealing ring being in slidable contact with an inner surface of the passageway of the annular dull probe portion so as to ensure that the sharp probes of the sharp portion contact the skin in substantial concentric relationship with the annular contact surface of the annular dull probe portion that contacts the skin.

9. The device of claim 8, further comprising:
a friction-free coating applied to the inner surface of the passageway so as to reduce friction between the sealing ring and the inner surface of the passageway.

10. The device of claim 1, wherein the guide portion of the central post includes an attachment feature for attaching the guide portion to a handle of a multiple test allergy skin testing device.

11. The device of claim 10, further including a handle of a multiple test allergy skin testing device.

12. A multiple test allergy skin testing device, the device to be used by pressing the device against skin, the device comprising:
a plurality of central posts, each central post having a sharp portion, a longitudinal portion, and a guide portion,
the sharp portion having one or more sharp probes extending longitudinally and being configured to administer an allergen to the skin,
the guide portion being configured to facilitate guiding the sharp portion towards the skin,
the longitudinal portion extending between the sharp portion and the guide portion;
a plurality of compressible annular pain-reduction structures, each compressible annular pain-reduction structure having an annular dull probe portion, a resilient compressible portion, and a base portion,
the annular dull probe portion initially extending beyond the sharp portion of the central post, and having a passageway allowing passage of the sharp portion therethrough, the annular dull probe portion being configured to activate a surrounding pain gate effect when pressed against the skin,
the resilient compressible portion being configured to compress along the longitudinal portion of the central post while the annular dull probe portion is pressed against the skin,
the base portion being configured to be pushed by the guide portion of the central post when the annular dull probe portion is pressed against the skin, thereby compressing the resilient compressible portion until the one or more sharp probes are pressed into the skin so as to administer the allergen to the skin; and
a gripping element configured to receive the plurality of central posts, the gripping element being for securely controlling the plurality of central posts and the respective plurality of compressible annular pain-reduction structures, so as to facilitate pressing the annular dull probe portion of each compressible annular pain-reduction structure, and then the sharp portion of each central post, into the skin using one press of the gripping element towards the skin.

13. The device of claim 12, wherein the resilient compressible portion of each compressible annular pain-reduction structure includes:
a plurality of bendable elements configured to resiliently bend when compressed.

14. The device of claim 12, wherein the resilient compressible portion of each compressible annular pain-reduction structure includes:
a bellows configured to resiliently shorten when compressed.

15. The device of claim 12, wherein the resilient compressible portion of each compressible annular pain-reduction structure includes:
a spring configured to resiliently shorten when compressed.

16. The device of claim 12, wherein each annular dull probe portion has a C-shaped cross-section.

17. The device of claim 12, wherein each annular dull probe portion includes one or more bumps on an annular surface of the annular dull probe portion that contacts the skin.

18. The device of claim 12, further comprising:
a sealing ring seated at a point along the sharp portion of the central post, the sealing ring being in slidable contact with an inner surface of the passageway of the annular dull probe portion so as to ensure that the sharp probes of the sharp portion contact the skin in substantial concentric relationship with the annular contact surface of the annular dull probe portion that contacts the skin.

19. The device of claim 18, further comprising:
a friction-free coating applied to the inner surface of the passageway so as to reduce friction between the sealing ring and the inner surface of the passageway.

20. The device of claim 12, wherein the guide portion of each central post includes an attachment feature for attaching the guide portion to the gripping element.

21. An allergy skin testing device, the device to be used by pressing the device against skin, the device comprising:
a central post having a sharp portion, a longitudinal portion, and a guide portion,
the sharp portion having one or more sharp probes extending longitudinally and being configured to administer an allergen to the skin,
the guide portion being configured to facilitate guiding the sharp portion towards the skin,
the longitudinal portion extending between the sharp portion and the guide portion; and
a compressible annular pain-reduction structure having an annular dull probe portion, and a resilient compressible portion,
the annular dull probe portion initially extending beyond the sharp portion of the central post, and having a passageway allowing passage of the sharp portion therethrough, the annular dull probe portion being configured to activate a surrounding pain gate effect when pressed against the skin,
the resilient compressible portion being configured to compress along the longitudinal portion of the central post while the annular dull probe portion is pressed against the skin.

22. The device of claim 21, wherein the compressible annular pain-reduction structure further includes:
a cover portion that covers the resilient compressible portion, the cover portion being attached to the annular dull probe portion of the compressible annular pain-reduction structure.

23. The device of claim 21, wherein the resilient compressible portion of the compressible annular pain-reduction structure includes:
a plurality of bendable elements configured to resiliently bend when compressed.

24. The device of claim 21, wherein the resilient compressible portion of the compressible annular pain-reduction structure includes:
a single bendable element configured to resiliently bend when compressed.

25. The device of claim 21, wherein the resilient compressible portion of the compressible annular pain-reduction structure includes:
a bellows configured to resiliently shorten when compressed.

26. The device of claim 21, wherein the resilient compressible portion of the compressible annular pain-reduction structure includes:
a spring configured to resiliently shorten when compressed.

27. The device of claim 21, wherein the annular dull probe portion includes one or more bumps on an annular surface of the annular dull probe portion that contacts the skin.

28. The device of claim 21, further comprising:
a friction-free coating applied to the inner surface of the passageway so as to reduce friction between the sharp portion and an inner surface of the passageway.

29. The device of claim 21, wherein the guide portion of the central post includes an attachment feature for attaching the guide portion to a handle of a multiple test allergy skin testing device.

30. The device of claim 29, further including a handle of a multiple test allergy skin testing device.
